(19) United States
(12) Patent Application Publication
(10) Pub. No.: US 2015/0088237 A1
(43) Pub. Date: Mar. 26, 2015

(54) LEAD EXTENSIONS FOR USE WITH ELECTRICAL STIMULATION SYSTEMS AND METHODS OF MAKING AND USING THE LEAD EXTENSIONS

(71) Applicant: Boston Scientific Neuromodulation Corporation, Valencia, CA (US)

(72) Inventors: David Ernest Wechter, Santa Clarita, CA (US); Joshua Dale Howard, Chatsworth, CA (US)

(21) Appl. No.: 14/494,405

(22) Filed: Sep. 23, 2014

Related U.S. Application Data
(60) Provisional application No. 61/881,184, filed on Sep. 23, 2013.

(51) Int. Cl. A61N 1/05

(52) U.S. Cl. CPC A61N 1/0539; A61N 1/0534
USPC 607/116

(57) ABSTRACT

An implantable lead extension includes an intermediate body element; a plurality of proximal tails attached to a proximal end portion of the intermediate body element; a plurality of terminals disposed along each of the plurality of proximal tails; and a connector assembly attached to a distal end portion of the intermediate body element. The connector assembly includes a plurality of connectors; each configured and arranged for electrically coupling with a different stimulation lead. Each connector has a connector housing defining a port for receiving a proximal end portion of a stimulation lead, and a plurality of connector contacts disposed in the connector housing. The connector contacts can couple to terminals of the stimulation lead when the proximal end portion of the stimulation lead is received by the port. Conductors extend along the longitudinal length of the intermediate body element and electrically couple the connector contacts to the terminals.

### CROSS-REFERENCE TO RELATED APPLICATIONS

This application claims the benefit under 35 U.S.C. §119(e) of U.S. Provisional Patent Application Ser. No. 61/881,184, filed Sep. 23, 2013, which is incorporated herein by reference.

### FIELD

The invention is directed to the area of electrical stimulation systems. The present invention is also directed to electrical stimulation systems having lead extensions suitable for concurrent use with multiple leads, as well as methods of making and using the lead extensions, leads, and electrical stimulation systems.

### BACKGROUND

Electrical stimulation can be useful for treating a variety of conditions. Deep brain stimulation can be useful for treating, for example, Parkinson's disease, dystonia, essential tremor, chronic pain, Huntington's disease, levodopa-induced dyskinesias and rigidity, bradykinesia, epilepsy and seizures, eating disorders, and mood disorders. Typically, a lead with a stimulating electrode at or near a tip of the lead provides the stimulation to target neurons in the brain. Magnetic resonance imaging (“MRI”) or computerized tomography (“CT”) scans can provide a starting point for determining where the stimulating electrode should be positioned to provide the desired stimulus to the target neurons.

After the lead is implanted into a patient's brain, electrical stimulus current can be delivered through selected electrodes on the lead to stimulate target neurons in the brain. Typically, the electrodes are formed into rings disposed on a distal portion of the lead. The ring electrodes project the stimulus current equally in every direction. Because of the ring shape of these electrodes, the stimulus current cannot be directed to one or more specific positions around the ring electrode (e.g., on one or more sides, or points, around the lead). Consequently, undirected stimulation may result in unwanted stimulation of neighboring neural tissue, potentially resulting in undesired side effects.

### BRIEF SUMMARY

In one embodiment, an implantable lead extension includes an intermediate body element having a proximal end portion, a distal end portion, and a longitudinal length. Additionally, the implantable lead extension includes multiple proximal tails attached to the proximal end portion of the intermediate body element. Multiple terminals are disposed along each of the proximal tails. The implantable lead extension also includes a connector assembly attached to the distal end portion of the intermediate body element. The connector assembly includes multiple connectors. Each connector includes a connector housing defining a port for receiving a proximal end portion of a stimulation lead. Each of the connectors also includes multiple connector contracts disposed in the connector housing. The connector contacts couple to terminals of the stimulation lead when the port receives the proximal end portion of the stimulation lead. The connector assembly further includes multiple conductors that extend along the longitudinal length of the intermediate body element and that electrically couple the connector contacts to the terminals.

In another embodiment, a method for implanting an electrical stimulation system includes providing the lead extension described above. A first stimulation lead is advanced into the skull of a patient. The first stimulation lead includes first electrodes disposed along a distal end portion of the first stimulation lead. In addition, the first stimulation lead includes multiple terminals disposed along the proximal end portion of the first stimulation lead. A second stimulation lead is advanced into the skull of the patient. The second stimulation lead includes second electrodes disposed along a distal end portion of the second stimulation lead and multiple second terminals disposed along a proximal end portion of the second stimulation lead. The first terminals of the first stimulation lead are coupled electrically to the connector assembly of the lead extension. The second terminals of the second stimulation lead are coupled electrically to the connector assembly of the lead extension. Terminals of the proximal tails of the lead extension are coupled to an implantable pulse generator.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of a device for brain stimulation, according to the invention;

FIG. 2 is a schematic diagram of radial current steering along various electrode levels along the length of a lead, according to the invention;

FIG. 3A is a perspective view of an embodiment of a portion of a lead having a plurality of segmented electrodes, according to the invention;

FIG. 3B is a perspective view of a second embodiment of a portion of a lead having a plurality of segmented electrodes, according to the invention;

FIG. 3C is a perspective view of a third embodiment of a portion of a lead having a plurality of segmented electrodes, according to the invention;

FIG. 3D is a perspective view of a fourth embodiment of a portion of a lead having a plurality of segmented electrodes, according to the invention;

FIG. 3E is a perspective view of a fifth embodiment of a portion of a lead having a plurality of segmented electrodes, according to the invention;

FIG. 3F is a perspective view of a sixth embodiment of a portion of a lead having a plurality of segmented electrodes, according to the invention;

FIG. 3G is a perspective view of a seventh embodiment of a portion of a lead having a plurality of segmented electrodes, according to the invention;

FIG. 4A is a schematic side view of one embodiment of a lead extension suitable for implanting into a patient, according to the invention;

FIG. 4B is a schematic longitudinal cross-sectional view of one embodiment of a portion of the lead extension of FIG. 4A, according to the invention;

FIG. 4C is a schematic transverse cross-sectional view of one embodiment of a connector assembly of the lead extension of FIG. 4A, according to the invention;

FIG. 4D is a schematic transverse cross-sectional view of another embodiment of a connector assembly of the lead extension of FIG. 4A, according to the invention;

FIG. 5 is a schematic side view of one embodiment of a portion of the lead extension of FIG. 4A with a strain relief feature disposed between a connector assembly and an intermediate body element of the lead extension, according to the invention;

FIG. 6A is a schematic side view of one embodiment of a portion of the lead extension of FIG. 4A and several anchoring units suitable for anchoring the lead extension to patient tissue, according to the invention;

FIG. 6B is a schematic side view of another embodiment of a portion of the lead extension of FIG. 4A and several anchoring units suitable for anchoring the lead extension to patient tissue, according to the invention;

FIG. 6C is a schematic side view of yet another embodiment of a portion of the lead extension of FIG. 4A and several anchoring units suitable for anchoring the lead extension to patient tissue, according to the invention;

FIG. 7A is a schematic transverse cross-sectional view of one embodiment of an intermediate body portion of a lead extension, the intermediate body portion including a conductor-carrying element defining conductor lumens, according to the invention;

FIG. 7B is a schematic transverse cross-sectional view of another embodiment of an intermediate body portion of a lead extension, the intermediate body portion including a conductor-carrying element defining conductor lumens, according to the invention;

FIG. 7C is a schematic transverse cross-sectional view of yet another embodiment of an intermediate body portion of a lead extension, the intermediate body portion including a conductor-carrying element defining conductor lumens, according to the invention;

FIG. 8 is a schematic side view of one embodiment of a portion of an intermediate body element of a lead extension, the intermediate body element including conductors configured into a single-layer coil and encased in an outer jacket, according to the invention;

FIG. 9 is a schematic side view of another embodiment of a portion of an intermediate body element of a lead extension, the intermediate body element including conductors configured into a multi-layer coil and encased in an outer jacket, according to the invention;

FIG. 10A is a schematic side view of one embodiment of a portion of an intermediate body element of a lead extension, the intermediate body element including coiled conductors encased in an outer jacket, the intermediate body element also including multi-lumen tubing disposed at opposing ends of the intermediate body element, according to the invention;

FIG. 10B is a schematic transverse cross-sectional view of one embodiment of the multi-lumen tubing of FIG. 10A, according to the invention;

FIG. 10C is a schematic transverse cross-sectional view of another embodiment of the multi-lumen tubing of FIG. 10A, according to the invention;

FIG. 10D is a schematic transverse cross-sectional view of yet another embodiment of the multi-lumen tubing of FIG. 10A, according to the invention;

FIG. 11 is a schematic side view of one embodiment of portions of multiple conductors configured into common-mode current suppression units, according to the invention; and

FIG. 12 is a schematic side view of one embodiment of a portion of an intermediate body element of a lead extension, the intermediate body element including two discrete groupings of conductors, each of the groupings of conductors configured into multiple common-mode current suppression units and extending side-by-side within a shared outer jacket, according to the invention.

### DETAILED DESCRIPTION

The invention is directed to the area of electrical stimulation systems. The present invention is also directed to electrical stimulation systems having lead extensions suitable for concurrent use with multiple leads, as well as methods of making and using the lead extensions, leads, and electrical stimulation systems.

A lead for deep brain stimulation may include stimulation electrodes, recording electrodes, or a combination of both. At least some of the stimulation electrodes, recording electrodes, or both are provided in the form of segmented electrodes that extend only partially around the circumference of the lead. These segmented electrodes can be provided in sets of electrodes, with each set having electrodes radially distributed about the lead at a particular longitudinal position. For illustrative purposes, the leads are described herein relative to use for deep brain stimulation, but it will be understood that any of the leads can be used for applications other than deep brain stimulation, including spinal cord stimulation, peripheral nerve stimulation, or stimulation of other nerves and tissues.

Suitable implantable electrical stimulation systems include, but are not limited to, at least one lead with one or more electrodes disposed on a distal end of the lead and one or more terminals disposed on one or more proximal ends of the lead. Leads include, for example, percutaneous leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; and 8,391,985; U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and U.S. patent applications Ser. Nos. 12/177,823; 13/667,953; and 13/750,725, all of which are incorporated by reference.

In at least some embodiments, a practitioner may determine the position of the target neurons using recording electrode(s) and then position the stimulation electrode(s) accordingly. In some embodiments, the same electrodes can be used for both recording and stimulation. In some embodiments, separate leads can be used; one with recording electrodes which identify target neurons, and a second lead with stimulation electrodes that replaces the first after target neuron identification. In some embodiments, the same lead may include both recording electrodes and stimulation electrodes or electrodes may be used for both recording and stimulation.

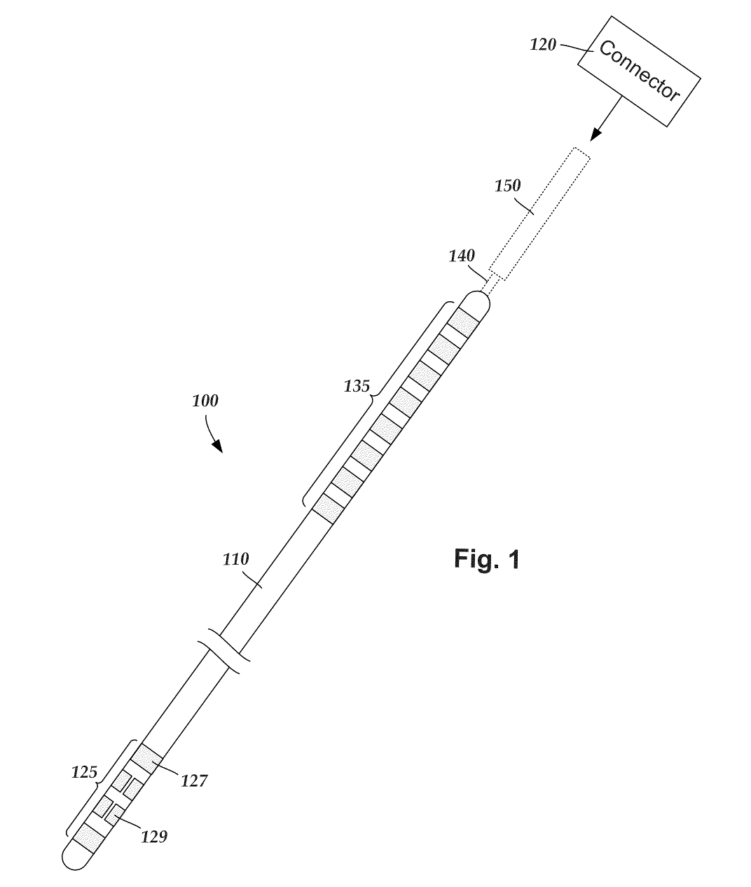

FIG. 1 illustrates one embodiment of a lead 100 for brain stimulation. The lead includes a lead body 110, a plurality of electrodes 125 disposed at least partially about a circumference of the lead body 110, a plurality of terminals 135, and a lead connector assembly 120 for connection of the electrodes to a control unit (not shown). A stylet 140 can be used for assisting in insertion and positioning of the lead body 110 in the patient's brain. In at least some embodiments, the stylet 140 is insertable into a stylet lumen (not shown) extending along a longitudinal length of the lead body 110. The stylet 140 can be made of a rigid material. Examples of suitable materials for the stylet include, but are not limited to, tungsten, stainless steel, and plastic. The stylet 140 may have a handle 150 to assist insertion into the lead body 110, as well as rotation of the stylet 140 and lead body 110. The connector assembly 120 fits over a proximal end portion of the lead body 110, preferably after removal of the stylet 140.

The control unit (not shown) is typically an implantable pulse generator that is implantable into a patient's body, for example, below the patient's clavicle area. The pulse generator can have eight stimulation channels, which may be independently programmable to control the magnitude of the current stimulus from each channel. In some cases, the pulse generator may have more or fewer than eight stimulation channels (e.g., 4-, 6-, 16-, 32-, or more stimulation channels). The control unit may have one, two, three, four, or more connector ports, for receiving the plurality of terminals 135 disposed along the proximal end portion of the lead body 110.

In one example of operation, access to the desired position in the brain can be accomplished by drilling a hole in the patient's skull or cranium with a cranial drill (commonly referred to as a burr), and coagulating and incising the dura mater, or brain covering. The lead body 110 is inserted into the cranium and brain tissue with the assistance of the stylet 140. The lead 100 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 100, retract the lead 100, or rotate the lead 100.

In some embodiments, measurement devices coupled to the muscles or other tissues stimulated by the target neurons, or a unit responsive to the patient or clinician, can be coupled to the control unit or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in tremor frequency or amplitude in response to stimulation of neurons. Alternatively, the patient or clinician may observe the muscle and provide feedback.

The lead 100 for deep brain stimulation can include stimulation electrodes, recording electrodes, or both. In at least some embodiments, the lead 100 is rotatable so that the stimulation electrodes can be aligned with the target neurons after the neurons have been located using the recording electrodes.

Stimulation electrodes may be disposed on the circumference of the lead body 110 to stimulate the target neurons. Stimulation electrodes may be ring-shaped so that current projects from each electrode equally in every direction from the position of the electrode along a length of the lead body 110. In at least some embodiments, the electrodes 125 include one or more ring electrodes 127 and one or more sets of segmented electrodes 129.

Ring electrodes 127 typically do not enable stimulus current to be directed from only a limited angular range around of the lead 100. Segmented electrodes 130, however, can be used to direct stimulus current to a selected angular range around the lead 100. When segmented electrodes 129 are used in conjunction with an implantable pulse generator that delivers constant current stimulus, current steering can be achieved to more precisely deliver the stimulus to a position around an axis of the lead body 110 (i.e., radial positioning around the axis of the lead body 110).

To achieve current steering, segmented electrodes can be utilized in addition to, or as an alternative to, ring electrodes. Though the following description discusses stimulation electrodes, it will be understood that all configurations of the stimulation electrodes discussed may be utilized in arranging recording electrodes as well.

The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead body 110 has a cross-sectional diameter of no more than 1.5 mm and may be in the range of 0.5 to 1.5 mm. In at least some embodiments, the lead body 110 has a length of at least 10 cm and the length of the lead body 110 may be in the range of 10 to 70 cm.

The ring electrodes 127 and segmented electrodes 129 may be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. In some embodiments, the ring electrodes 127 and the segmented electrodes 129 are made of the same materials. In other embodiments, the ring electrodes 127 and the segmented electrodes 129 are made of different materials. Preferably, the ring electrodes 127 and segmented electrodes 129 are made of a material that is biocompatible and does not substantially corrode under expected operating conditions in the operating environment for the expected duration of use.

Each of the electrodes can be either used or unused (OFF). When the electrode is used, the electrode can be used as an anode or cathode and carry anodic or cathodic current. In some instances, an electrode might be an anode for a period of time and a cathode for a period of time.

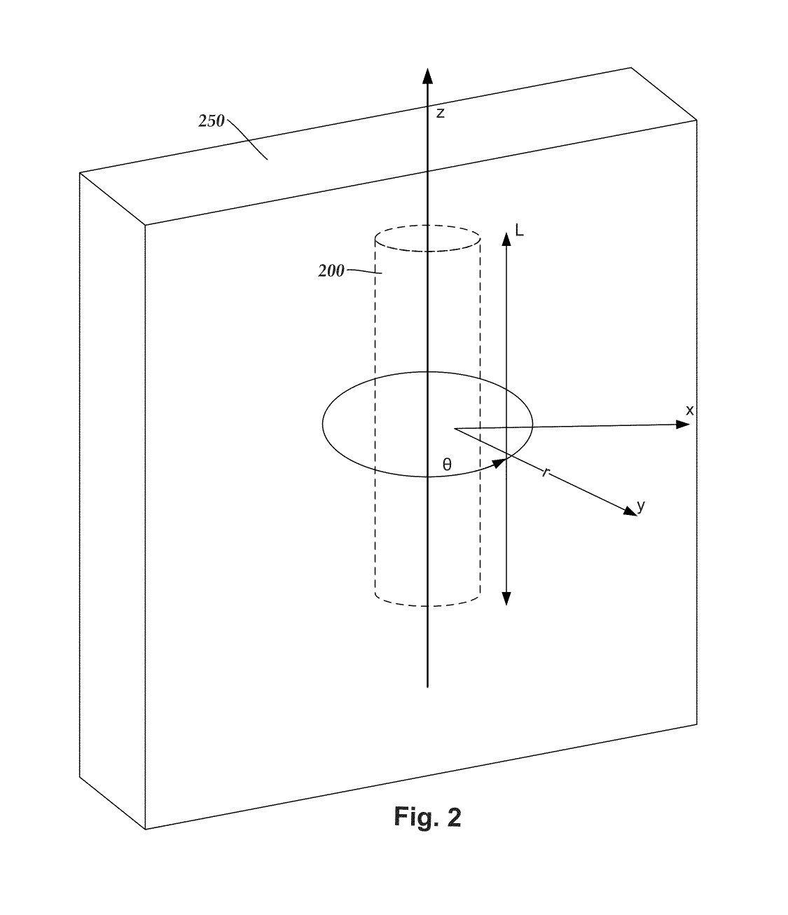

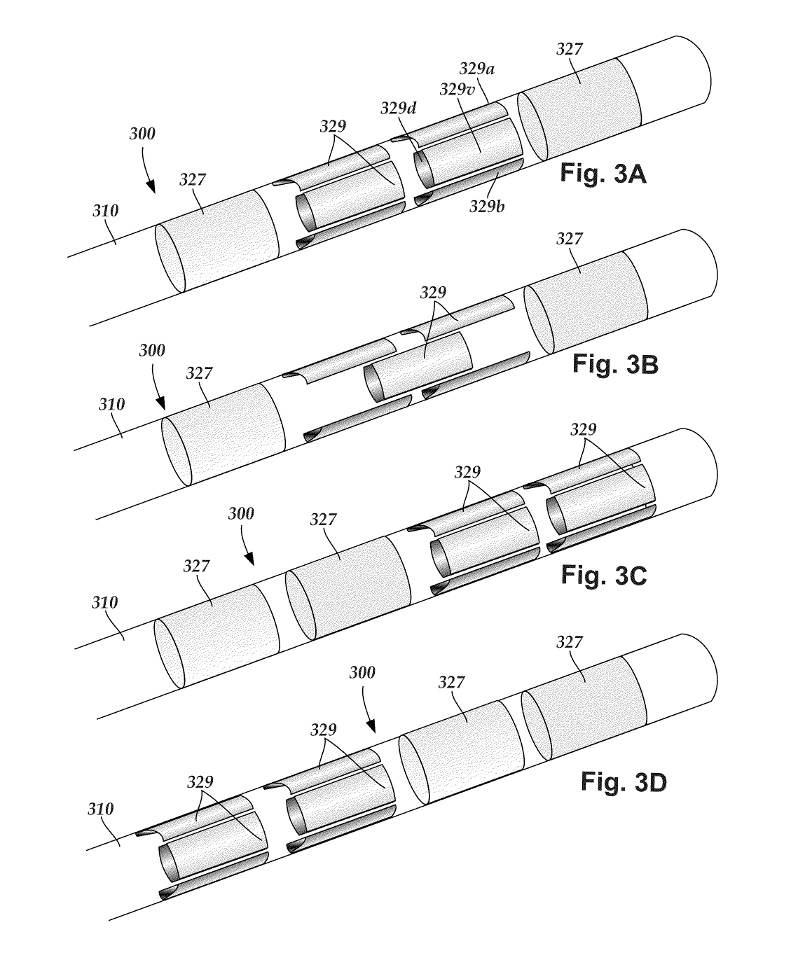

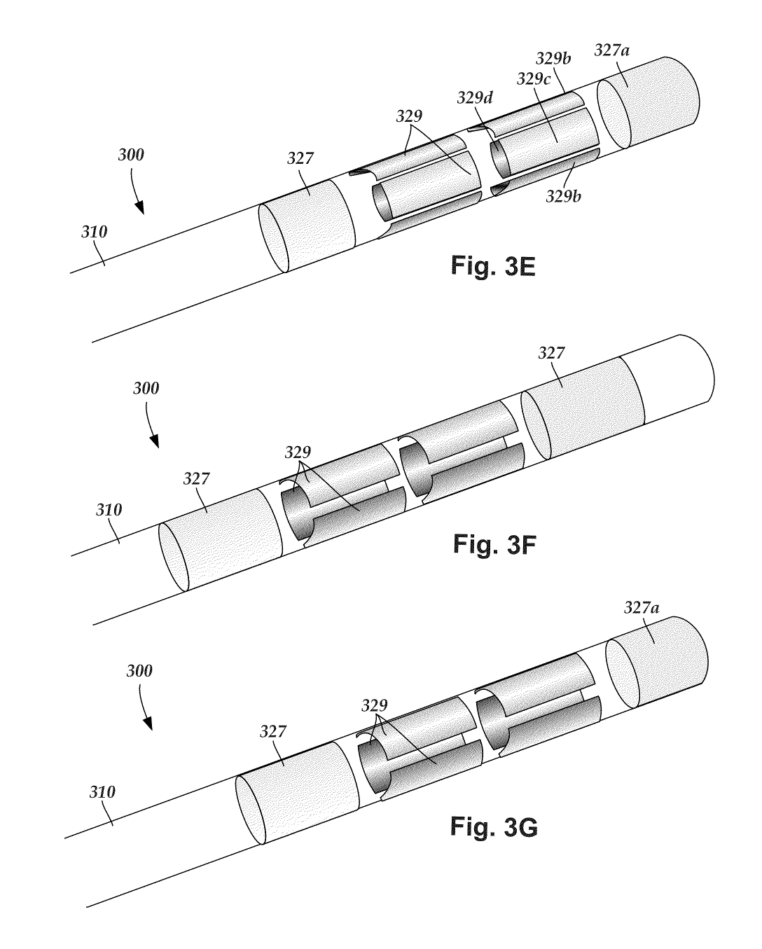

Stimulation electrodes in the form of ring electrodes 127 may be disposed along any part of the lead body 110, usually along a distal end portion of the lead body 110. In FIG. 1, the lead body 110 includes two ring electrodes 127. Any number of ring electrodes 127 may be disposed along the length of the lead body 110 including, for example, one, two three, four, five, six, seven, eight, nine, ten, eleven, twelve, thirteen, fourteen, fifteen, sixteen, or more ring electrodes 127. It will be understood that any number of ring electrodes 127 may be disposed along the length of the lead body 110. In some embodiments, the ring electrodes 127 are substantially cylindrical and wrap around the entire circumference of the lead body 110. In some embodiments, the outer diameters of the ring electrodes 127 are substantially equal to the outer diameter of the lead body 110. The length of the ring electrodes 127 may vary according to the desired treatment and the location of the target neurons. In some embodiments the length of the ring electrodes 127 are less than or equal to the diameters of the ring electrodes 127. In other embodiments, the lengths of the ring electrodes 127 are greater than the diameters of the ring electrodes 127. The distal-most ring electrode 120 may be a tip electrode (see e.g., tip electrode 327a of FIG. 3E) which covers most, or all, of the distal tip of the lead body 110.

Deep brain stimulation leads may include one or more sets of segmented electrodes 129. Segmented electrodes 129 may provide for superior current steering than ring electrodes 127 because target structures in deep brain stimulation are not typically symmetric about the axis of the distal electrode array. Instead, a target may be located on one side of a plane running through the axis of the lead. By a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Patent Application Publication Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321, all of which are incorporated herein by reference.

In FIG. 1, the lead 100 includes a plurality of segmented electrodes 129. Any number of segmented electrodes 129 may be disposed along the lead 100 including, for example, one, two three, four, five, six, seven, eight, nine, ten, eleven, twelve, thirteen, fourteen, fifteen, sixteen, or more segmented electrodes 129. It will be understood that any number of segmented electrodes 129 may be disposed along the lead 100. A segmented electrode 130 typically extends only 75%, 67%, 60%, 50%, 40%, 33%, 25%, 20%, 17%, 15%, or less around the circumference of the lead body 110.

The segmented electrodes 129 may be grouped into sets of segmented electrodes 129, where each set is disposed around a circumference of the lead body 110 at a particular longitudinal portion of the lead body 110. The lead body 110 may have any number segmented electrodes 129 in a given set of segmented electrodes 129. The lead body 110 may have one, two, three, four, five, six, seven, eight, or more segmented electrodes 129 in a given set. In at least some embodiments, each set of segmented electrodes 129 of the lead body 110 contains the same number of segmented electrodes 129. The segmented electrodes 129 disposed on the lead body 110 may include a different number of electrodes than at least one other set of segmented electrodes 129 disposed on the lead body 110.

The segmented electrodes 129 may vary in size and shape. In some embodiments, the segmented electrodes 129 are all of the same size, shape, diameter, width, or area or any combination thereof. In some embodiments, the segmented electrodes 129 of each circumferential set (or even all segmented electrodes 129 disposed on the lead body 110) may be identical in size and shape.

Each set of segmented electrodes 129 may be disposed around the circumference of the lead body 110 to form a substantially cylindrical shape around the lead body 110. The spacing between individual electrodes of a given set of the segmented electrodes 129 may be the same, or different from, the spacing between individual electrodes of another set of segmented electrodes 129 on the lead body 110. In at least some embodiments, equal spaces, gaps, or cutouts are disposed between each segmented electrode 130 around the circumference of the lead body 110. In other embodiments, the spaces, gaps, or cutouts between the segmented electrodes 129 may differ in size or shape. In other embodiments, the spaces, gaps, or cutouts between segmented electrodes 129 may be uniform for a particular set of the segmented electrodes 129, or for all sets of the segmented electrodes 129. The sets of segmented electrodes 129 may be positioned in irregular or regular intervals along a length the lead body 110.

Conductor wires that attach to the ring electrodes 127 or segmented electrodes 129 extend along the lead body 110. These conductor wires may extend through the material of the lead body 110 or along one or more lumens defined by the lead body 110, or both. The conductor wires are presented at a connector (via terminals) for coupling of the electrodes 127, 129 to a control unit (not shown).

When the lead body 110 includes both ring electrodes 127 and segmented electrodes 129, the ring electrodes 127 and the segmented electrodes 129 may be arranged in any suitable configuration. For example, when the lead body 110 includes two sets of ring electrodes 127 and two sets of segmented electrodes 129, the ring electrodes 127 can flank the two sets of segmented electrodes 129 (see e.g., FIG. 1). Alternately, the two sets of ring electrodes 127 can be disposed proximal to the two sets of segmented electrodes 129 (see e.g., FIG. 3C), or the two sets of ring electrodes 127 can be disposed distal to the two sets of segmented electrodes 129 (see e.g., FIG. 3D). One of the ring electrodes 127 can be a tip electrode (see, tip electrode 327a of FIGS. 3E and 3G). It will be understood that other configurations are possible as well (e.g., alternating ring and segmented electrodes, or the like).

By varying the location of the segmented electrodes 129, different coverage of the target neurons may be selected. For example, the electrode arrangement of FIG. 3C may be useful if the physician anticipates that the neural target will be closer to a distal tip of the lead body 110, while the electrode arrangement of FIG. 3D may be useful if the physician anticipates that the neural target will be closer to a proximal end portion of the lead body 110.

Any combination of ring electrodes 127 and segmented electrodes 129 may be disposed on the lead body 110. For example, the lead body 110 may include a first ring electrode 120, two sets of segmented electrodes; each set formed of four segmented electrodes 129, and a final ring electrode 120 at the end of the lead body 110. This configuration may simply be referred to as a 1-4-4-1 (FIGS. 3A and 3E) configuration. It may be useful to refer to the electrodes with this shorthand notation. Thus, the embodiment of FIG. 3C may be referred to as a 1-1-4-4 configuration, while the embodiment of FIG. 3D may be referred to as a 4-4-1-1 configuration. The embodiments of FIGS. 3F and 3G can be referred to as a 1-3-3-1 configuration. Other electrode configurations include, for example, a 2-2-2-2 configuration, where four sets of segmented electrodes are disposed on the lead body 110, and a 4-4 configuration, where two sets of segmented electrodes, each having four segmented electrodes 129 are disposed on the lead body 110. The 1-3-3-1 electrode configuration of FIGS. 3F and 3G has two sets of segmented electrodes, each set containing three electrodes disposed around the circumference of the lead body 110, flanked by two ring electrodes (FIG. 3F) or a ring electrode and a tip electrode (FIG. 3G). In some embodiments, the lead body 110 includes 16 electrodes. Possible configurations for a 16-electrode lead body 110 include, but are not limited to 4-4-4-4; 8-8; 3-3-3-3-3-1 (and all rearrangements of this configuration); and 2-2-2-2-2-2-2-2.

FIG. 2 is a schematic diagram to illustrate radial current steering along various electrode levels along the length of the lead 200. While conventional lead configurations with ring electrodes are only able to steer current along the length of the lead 200 (the z-axis), the segmented electrode configuration is capable of steering current in the x-axis, y-axis as well as the z-axis. Thus, the centroid of stimulation may be steered in any direction in the three-dimensional space surrounding the lead 200. In some embodiments, the radial distance, r, and the angle θ around the circumference of the lead 200 may be dictated by the percentage of anodic current (recognizing that stimulation predominantly occurs near the cathode, although strong anodes may cause stimulation as well) introduced to each electrode. In at least some embodiments, the configuration of anodes and cathodes along the segmented electrodes allows the centroid of stimulation to be shifted to a variety of different locations along the lead 200.

As can be appreciated from FIG. 2, the centroid of stimulation can be shifted at each level along the length of the lead 200. The use of multiple sets of segmented electrodes at different levels along the length of the lead 200 allows for three-dimensional current steering. In some embodiments, the sets of segmented electrodes are shifted collectively (i.e., the centroid of simulation is similar at each level along the length of the lead 200). In at least some other embodiments, each set of segmented electrodes is controlled independently. Each set of segmented electrodes may contain two, three, four, five, six, seven, eight, or more segmented electrodes. It will be understood that different stimulation profiles may be produced by varying the number of segmented electrodes at each level. For example, when each set of segmented electrodes includes only two segmented electrodes, uniformly distributed gaps (inability to stimulate selectively) may be formed in the stimulation profile. In some embodiments, at least three segmented electrodes 230 in a set are utilized to allow for true 360° selectivity.

As previously indicated, the foregoing configurations may also be used while utilizing recording electrodes. In some embodiments, measurement devices coupled to the muscles or other tissues stimulated by the target neurons or a unit responsive to the patient or clinician can be coupled to the control unit or microdrive motor system. The measurement device, user, or clinician can indicate a response by the target muscles or other tissues to the stimulation or recording electrodes to further identify the target neurons and facilitate positioning of the stimulation electrodes. For example, if the target neurons are directed to a muscle experiencing tremors, a measurement device can be used to observe the muscle and indicate changes in tremor frequency or amplitude in response to stimulation of neurons. Alternatively, the patient or clinician may observe the muscle and provide feedback.

The reliability and durability of the lead 200 will depend heavily on the design and method of manufacture. Fabrication techniques discussed below provide methods that can produce manufacturable and reliable leads 200.

Returning to FIG. 1, when the lead body 110 includes a plurality of sets of segmented electrodes 129, it may be desirable to form the lead body 110 such that corresponding electrodes of different sets of segmented electrodes 129 are circumferentially aligned with one another along the length of the lead body 110 (see e.g., the segmented electrodes 129 shown in FIG. 1). Circumferential alignment between corresponding electrodes of different sets of segmented electrodes 129 along the length of the lead body 110 may reduce uncertainty as to the location or orientation between corresponding segmented electrodes of different sets of segmented electrodes. Accordingly, it may be beneficial to form electrode arrays such that corresponding electrodes of different sets of segmented electrodes along the length of the lead body 110 are circumferentially aligned with one another and do not radially shift in relation to one another during manufacturing of the lead body 110.

In other embodiments, individual electrodes in the two sets of segmented electrodes 129 are staggered (see, FIG. 3B) relative to one another along the length of the lead body 110. In some cases, the staggered positioning of corresponding electrodes of different sets of segmented electrodes along the length of the lead body 110 may be designed for a specific application.

Segmented electrodes can be used to tailor the stimulation region so that, instead of stimulating tissue around the circumference of the lead 200 as would be achieved using a ring electrode, the stimulation region can be directionally targeted. In some instances, it is desirable to target a parallelepiped (or slab) region 250 that contains the electrodes of the lead 200, as illustrated in FIG. 2. One arrangement for directing a stimulation field into a parallelepiped region uses segmented electrodes disposed on opposite sides of a lead 200.

FIGS. 3A-3G illustrate leads 300 with segmented electrodes 329, optional ring electrodes 327 or tip electrodes 327a, and a lead body 310. The sets of segmented electrodes 329 include either two (FIG. 3B), three (FIGS. 3F and 3G), or four (FIGS. 3A, 3C, and 3D) or any other number of segmented electrodes 329 including, for example, five, six, or more.

Any other suitable arrangements of segmented electrodes can be used. As an example, arrangements in which segmented electrodes are arranged helically with respect to each other can be used. One embodiment includes a double helix.

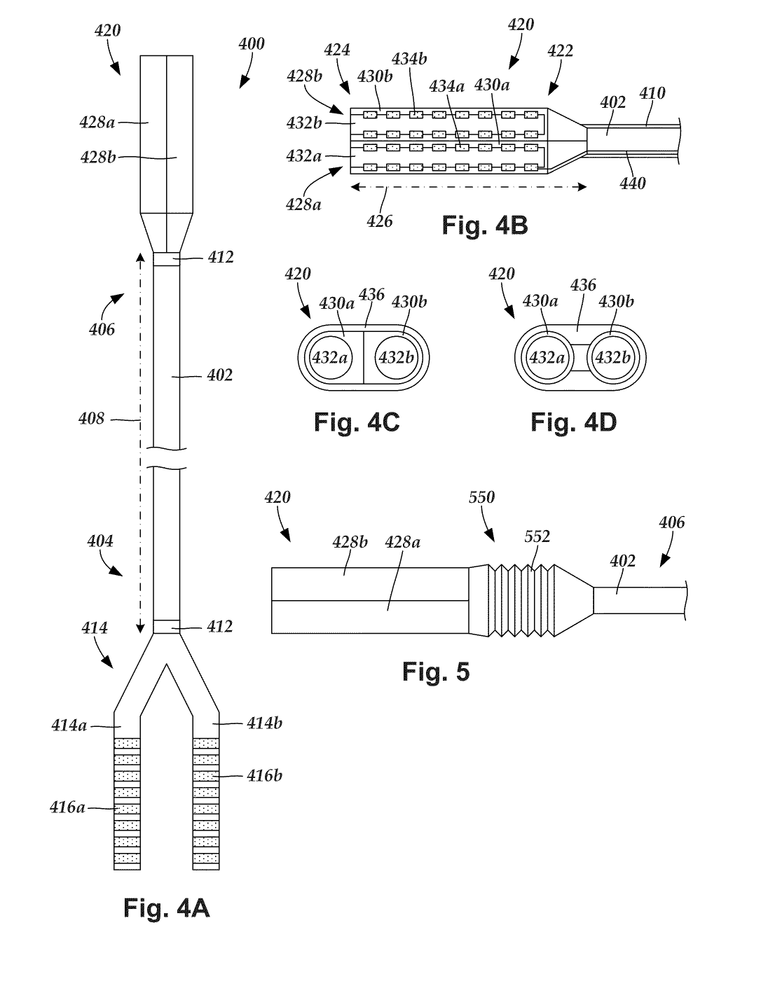

Turning to FIGS. 4A-4D, leads are often connected to the control unit (which provides stimulation energy) via lead extensions. In some instances, a patient is stimulated using two or more leads (e.g., bi-lateral stimulation, or the like). Each of the multiple leads is typically coupled to the control unit using a different lead extension. It may be advantageous to reduce the number of lead extensions implanted into a patient to reduce procedure time, simplify implantation procedures, reduce financial cost associated with each lead extension, reduce the number of parts that may potentially fail during operation, simplify the stimulation system, reduce the number of implanted structures disposed in a patient, and the like.

Lead extensions may be prone to migrating over time. Migration may be detrimental, for example, by moving the electrodes of the lead out of range of the target stimulation location, thereby disrupting therapy. Some regions of the body may be particularly susceptible to migration. For example, in the case of deep brain stimulation, the control unit may be implanted in the patient's upper chest. In which case, the lead extension typically extends along the patient's neck. Movement of the patient's neck may cause undesired migration of the lead extension over time. In at least some instances, it may be desirable to anchor a lead extension to patient tissue to prevent lead migration. In at least some instances, it may be desirable to promote tissue in-growth along outer surfaces of the lead extension in order to facilitate lead anchoring.

Lead extensions may be exposed to strain during patient movement (e.g., neck craning, or the like), which may cause a lead extension to undesirable separate from a lead or a control unit, or even to fail. It may be advantageous to design a lead extension to provide strain relief to absorb strain placed upon one or more portions of the lead extension during patient movement.

In some cases, a patient with an implanted electrical stimulation system may be exposed to transient RF current (e.g., via an MRI procedure, or the like) that causes common-mode coupling of current along the lead, the lead extension, the control unit, or some combination thereof. Such common-mode coupling of energy may cause undesired stimulation and burning of patient tissue, or failure of the implanted electrical stimulation system, or both. It may be advantageous to design lead extensions with the ability to prevent common-mode coupling of current from occurring, or to disrupt such coupled current, or to reduce the impact of such coupled current on patient tissue and electrical systems.

As herein described, a lead extension includes a connector assembly designed to receive multiple leads and couple each of the received leads to a control unit. In at least some embodiments, the lead extension is designed for anchoring to patient tissue, either by one or more anchoring units, tissue in-growth, or both. In at least some embodiments, the lead extension includes a strain relief. In at least some embodiments, the lead extension is designed for reducing the effects of undesired common-mode coupling of current resulting from exposure of the patient to transient RF energy. Under at least some conditions, it may be safe for a patient to undergo an MRI procedure with the lead extension implanted in the patient.

FIGS. 4A-4D illustrate a lead extension 400 suitable for implanting into a body of a patient. FIG. 4A illustrates, in side view, one embodiment of the lead extension 400. FIG. 4B illustrates, in longitudinal cross-sectional view, one embodiment of a distal end portion of the lead extension 400. FIG. 4C illustrates, in transverse cross-sectional view, one embodiment of a distal end portion of the lead extension 400. FIG. 4D illustrates, in transverse cross-sectional view, another embodiment of a distal end portion of the lead extension 400.

The lead extension 400 includes an intermediate body element 402, one or more proximal tails 414, and a connector assembly 420. The intermediate body element 402 is an elongate member having a proximal end portion 404, a distal end portion 406, and a longitudinal length 408. The proximal tails 414 are attached to the intermediate body element 402 along the proximal end portion 404 of the intermediate body element 402. The connector assembly 420 is attached to the intermediate body element 402 along the distal end portion 406 of the intermediate body element 402. The lead extension 400 may have a length suitable to coupling proximal end portions of leads, such as lead (100 of FIG. 1), with distal end portions disposed in opposing sides of the patient's brain, to the control unit (a pulse generator) disposed posterior to the patient's neck, for example, in the clavicle region.

In at least some embodiments, the intermediate body element 402 has a longer length than the proximal tails 414. The intermediate body element 402 can be flexible and stretchable to allow the lead extension 400 to move along with the patient's movements during operation. Suitable materials for forming the intermediate body element 402 may include, but not limited to, plastic, such as, silicone rubber, thermoplastic polyurethane, polytetrafluoroethylene, PEEK, polyvinylidene fluoride, polyethylene terephthalate, urethane-silicone copolymers polyimide, polyamide, or the like or combinations thereof.

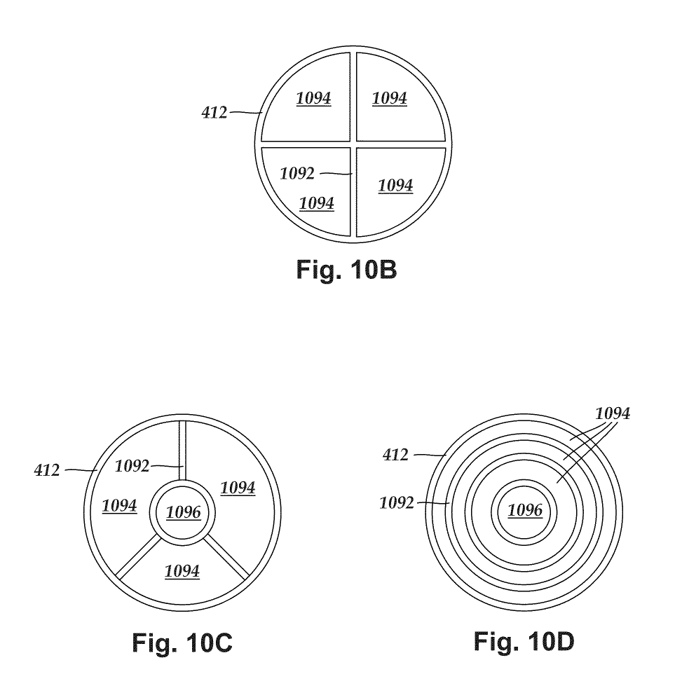

An optional multi-lumen tubing 412 may be disposed along one or more opposing ends of the intermediate body element 402, at the junctions between the intermediate body element 402 and proximal tails 414, or at the junction between the intermediate body element 402 and connector assembly 420, or both. The multi-lumen tubing is described in detail below with reference to FIGS. 10A-10D.

The lead extension 400 can include any suitable number of proximal tails 414 including, for example, two, three, four, five, six, seven, eight, or more proximal tails. The proximal tails 414a and 414b are designed for coupling to a control unit. Terminals are disposed along one or more of the proximal tails 414. The terminals are configured for coupling to a connector (e.g., of the control unit, or the like). In FIG. 4A, the lead extension is shown having a first proximal tail 414a and a second proximal tail 414b. In FIG. 4A, multiple first terminals 416a are shown disposed along the first proximal tail 414a, and multiple second terminals 416b are shown disposed along the second proximal tail 414b.

The connector assembly 420 includes a proximal end portion 422, a distal end portion 424, and a longitudinal length 426. The connector assembly 420 includes multiple connectors 428, such as a first connector 428a and a second connector 428b. In some embodiments, the two connectors 428a and 428b are identical; and in other embodiments, the two connectors 428a and 428b are not identical.

The first connector 428a includes a first connector housing 430a extending along the longitudinal length 426 of the connector assembly 420. A first connector port 432a is defined by the first connector housing 430a and opens along the distal end portion 424 of the connector assembly 420. Multiple first connector contacts, such as first connector contact 434a, are disposed within the first connector housing 430a. Similarly, the second connector 428b includes a second connector housing 430b extending along the longitudinal length 426 of the connector assembly 420. A second connector port 432b is defined by the second connector housing 430b and opens along the distal end portion 424 of the connector assembly 420. Multiple second connector contacts, such as second connector contact 434b, are disposed within the second connector housing 430b. The ports 432a and 432b form female connectors with suitable cross-sections for each receiving a different proximal end portion of a lead, such as the lead (100 of FIG. 1). The connector contacts 434a and 434b are coupled to conductors, such as conductor 440, which extend along the lead extension and couple to the terminals 416a and 416b.

The connector housings 430a and 430b may be coupled to one another along one or more locations along the longitudinal length 426 of the connector assembly 420. In at least some embodiments, the connector housings 430a and 430b are attached to one another along the entire longitudinal length 426 of the connector assembly 420. The connector housings 430a and 430b can have any suitable transverse cross-sectional shape. In FIG. 4C, the connector housings 430a and 430b are shown having opposing D-shaped cross-sections with the ports 432a and 432b embedded within. In FIG. 4D, the connector housings 430a and 430b are shown having circular cross-sections coupled to one another with ports 432a and 432b embedded within.

The connector assembly 420 is sized to define ports for receiving proximal end portions of leads, such as lead (100 of FIG. 1). The connector assembly 420 can have any suitable transverse cross-sectional shape including, for example, round, oval, oblong, rectangular (with square or rounded corners), or the like.

The connector housing 430a and 430b may be made using non-conductive, biocompatible material including, for example, silicone, polyurethane, polyetheretherketone (“PEEK”), epoxy, and the like or combinations thereof. The connector contacts 434 may be formed using any conductive, biocompatible material such as, but not limited to, metals, alloys, conductive polymers, conductive carbon, and the like, as well as combinations thereof.

Optionally, an outer jacket 410 is disposed over longitudinal surfaces of the intermediate body element 402. Optionally, an outer jacket 436 is disposed over longitudinal surfaces of the connector assembly 420. The outer jackets 410 and 436 can be formed either from the same material(s) or from different materials. The outer jackets 410 and 436 can be a flexible polymeric coating made of materials, such as thermoplastic polyurethane, polyethylene, silicone, polyethylene terephthalate, or the like.

In at least some embodiments, one or more of the outer jacket 410 and the outer jacket 436 have a porous surface for promoting tissue ingrowth. The tissue ingrowth may facilitate anchoring of the lead extension 400 to patient tissue. As an example, in at least some embodiments a polyethylene terephthalate mesh may be included in the outer jacket 410, the outer jacket 436, or both. The polyethylene terephthalate mesh facilitates tissue ingrowth. In addition, the polyethylene terephthalate mesh is hydrophilic and wets easily in tissue fluids, thereby promoting tissue ingrowth. Further, when outer jackets are formed from meshed structures, the contact area of the lead extension 400 with patient tissue is larger as compared to the longitudinal surfaces of the intermediate body element 402, thereby potentially improving heat dissipation, as compared to non-porous surfaces.

Turning to FIG. 5, the lead extension 400 may, in at least some embodiments, be implanted along a portion of the patient (e.g., the patient's neck) that experiences strain caused by the patient's movements. Such movement may cause one or more ill-effects including, for example, undesired migration of the lead extension (and the one or more leads coupled thereto), uncoupling of the lead extension from the one or more coupled leads, failure along the electrical stimulation system, reduced patient movement, or the like or combinations thereof.

To address these challenges, the lead extension 400 may include one or more strain relief features to dampen strain placed along the lead extension 400 during implantation of the lead extension 400. FIG. 5 illustrates, in side view, one embodiment of a strain relief feature 500. The strain relief feature 550 can be disposed along any suitable portion of the lead extension 400. In at least some embodiments, the strain relief feature 550 is disposed between the connector assembly 420 and the intermediate body element 402. The strain relief feature 550 can be formed as any suitable structure for dampening strain including, for example, one or more coils, bends, zigzags, crimps, arches, sinusoids, hooks, wiggles, squiggles, arcs, curls, rings, ringlets, waves, undulations, serpentines, loops, jumbles, knots, overlapping regions, or the like or combinations thereof.

In FIG. 5, the strain relief feature 550 is shown as an accordion-like structure having a bellows (e.g., pleated) element 552 for axially expanding and contracting. For example, the accordion or bellows element 552 can include concentric rings of a flexible material, such as thermoplastic polyurethane, to allow for axial deformation at the junction of the intermediate body element 402 and connector assembly 420. Suitable materials for strain relief feature 550 can include plastics, such as, silicone rubber, Thermoplastic polyurethane, polytetrafluoroethylene, PEEK, polyvinylidene fluoride, polyethylene terephthalate, urethane-silicone copolymers polyimide, polyamide, or the like.

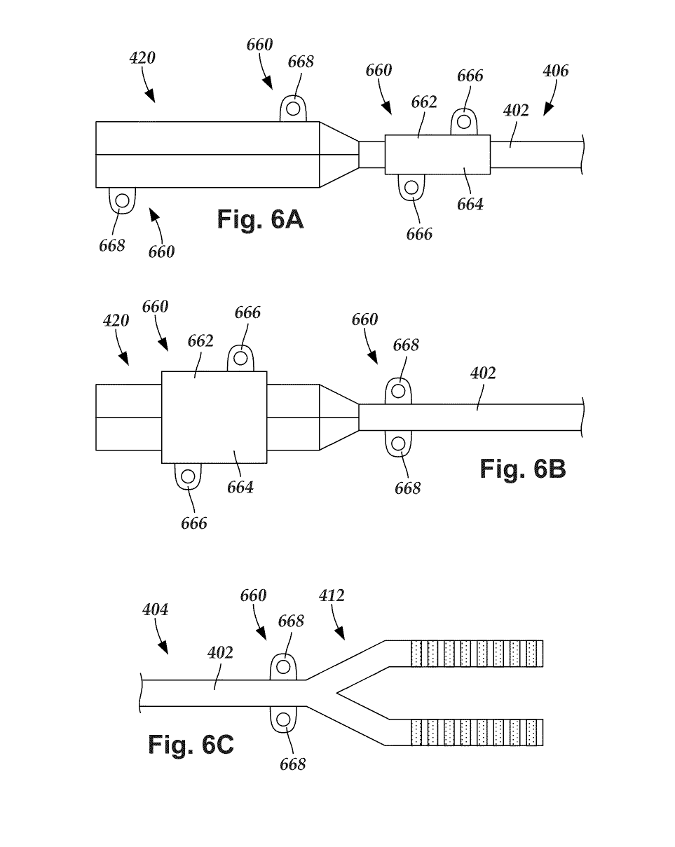

Turning to FIGS. 6A-6C, one or more anchoring elements may be used to anchor the lead extension 400 to patient tissue at a desired location. In some embodiments, the lead extension 400 is anchored to patient tissue along a single location along the length of the lead extension 400. In other embodiments, the lead extension 400 is anchored to patient tissue at along multiple locations along the length of the lead extension 400. Any suitable anchoring unit may be used to anchor the lead extension 400 to patient tissue.

FIGS. 6A-6C illustrate, in side view, several different embodiments of one or more anchoring units 660 disposed along one or more portions of the lead extension 400. The anchoring units 660 include a suture sleeve 662 that can be disposed over a portion of the lead extension 400, and eyelets 668 attached directly to one or more portions of the lead extension 400. The suture sleeve 662 includes a suture sleeve body 664 and a number of suture-sleeve eyelets 666 coupled to the suture sleeve body 664. The suture sleeve 662 and the eyelets 668 can be configured to receive sutures, staples, or the like, for anchoring the lead extension 400 to patient tissue. The suture sleeve body 664 provides support for the suture sleeve eyelets 666 for anchoring and may define suture channels (not shown) for wrapping sutures around the sleeve 662 to anchor the lead extension 400 into the tissue of the patient.

The suture sleeve 662 and the eyelets 668 can be used together along different portions of the lead extension 400, or one can be used exclusively along one or more portions of the lead extension 400. In FIG. 6A, eyelets 668 are shown disposed along the connector assembly 420 and a suture sleeve 662 is shown disposed along the intermediate body element 402. In FIG. 6B, a suture sleeve 662 is shown disposed along the connector assembly 420 and eyelets 668 are shown disposed along the intermediate body element 402. In FIG. 6C, eyelets 668 are shown disposed along the proximal end portion 404 of the intermediate body element 402. It will be understood that a suture sleeve 662 can be disposed along the proximal end portion 404 of the intermediate body element 402 in addition to, or in lieu of, the eyelets 668.

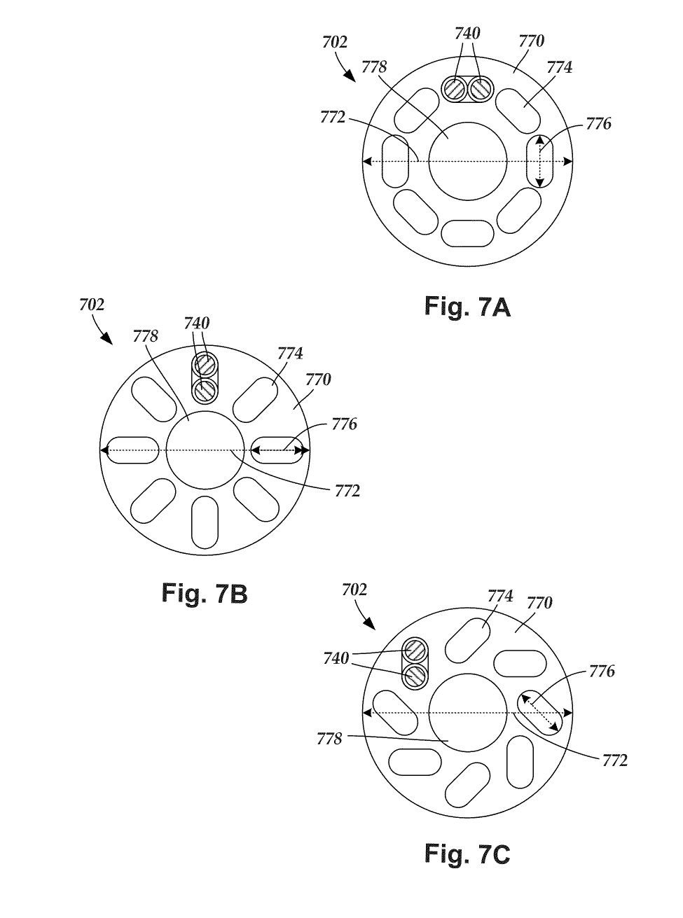

Turning to FIGS. 7A-12, the lead extension 400 is suitably designed to receive multiple conductors extending between the connector contacts and the terminals. The conductors can extend along the lead extension in any suitable manner. In at least some embodiments, the conductors extend along one or more conductor-carrying elements disposed along the intermediate body element.

FIGS. 7A-7C are schematic transverse cross-sectional views of three different embodiments of conductor-carrying element 770 suitable for extending along an intermediate body element 702. The conductor-carrying element 770 defines multiple conductor lumens 774. The conductor lumens 774 are each configured for receiving one or more conductors 740. Insulation is typically disposed around the conductors 740 to prevent short-circuiting.

In FIGS. 7A-7C, the conductor lumens 774 are shown each configured for receiving two conductors 740. It will be understood that the conductor lumens 774 can be configured for receiving any suitable number of conductors 740 including, for example, one, two, three, four, five, six, seven, eight, or more conductors. In some embodiments, each of the conductor lumens 774 is configured for receiving the same number of conductors. In other embodiments, at least one of the conductors is configured for receiving a different number of conductors from at least one other of the conductor lumens 774.

Optionally, the conductor-carrying element 770 defines a stylet lumen 778 for receiving a stylet (see e.g., 140 in FIG. 1) for facilitating implantation of the lead extension by increasing rigidity to the intermediate body portion 702 during insertion of the lead extension 400 into the patient.

In at least some embodiments, the conductor lumens 774 are shaped such that the conductor lumens 774 include a major transverse axis 776 that is longer than any other transverse axis of the conductor lumens 774. Any particular conductor lumen 774 of the multiple conductor lumens 774 can be arranged in any suitable orientation relative to a transverse diameter 772 of the conductor-carrying element 770 extending through that conductor lumen 774.

In FIG. 7A, the conductor lumens 774 are shown arranged such that the major transverse axis 776 of a particular conductor lumen 774 of the conductor lumens 774 is oriented perpendicular to the diameter 772 extending through that conductor lumen 774. In FIG. 7B, the conductor lumens 774 are shown arranged such that the major transverse axis 776 of a particular conductor lumen 774 of the conductor lumens 774 is oriented parallel to the diameter 772 extending through that conductor lumen 774. In FIG. 7C, the conductor lumens 774 are arranged such that the major transverse axis 776 of a particular conductor lumen 774 of the conductor lumens 774 is oriented neither parallel nor perpendicular to the diameter 772 extending through that conductor lumen 774.

The conductor-carrying element 770 can be formed as a single-piece component or as a multi-piece component. The conductor-carrying element 770 can be formed using various suitable material(s). For example, the conductor-carrying element 770 can be formed from one or more thermosetting polymers, thermoplastic polymers (e.g., polyurethane, or the like), silicone, or the like or combinations thereof. Further, the conductor-carrying element 770 can be formed in any suitable manner. For example, the conductor-carrying element 770 can be extruded. In some cases, the conductor-carrying element 770 can be twisted as the conductor-carrying element 770 is being extruded, or after extrusion. The conductor-carrying element 770 can be formed such that the conductor lumens 774 are in substantially straight configurations.

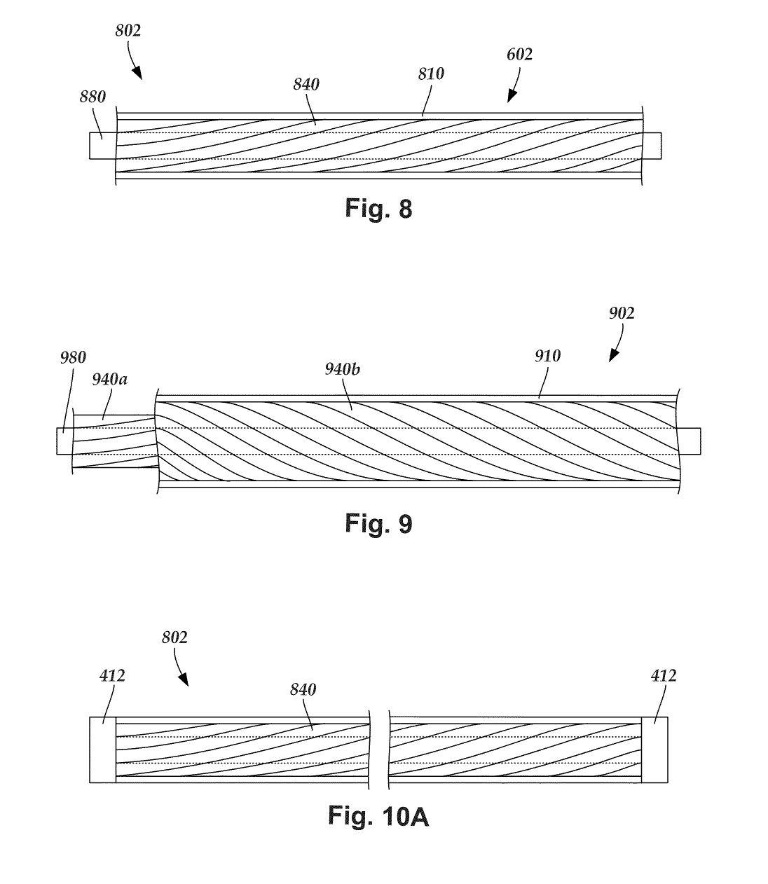

Turning to FIG. 8, in at least some embodiments, in addition to or in lieu of extending along a conductor-carrying element, the conductors are formed into coils that extend along at least a portion of the longitudinal length of the intermediate body element. FIG. 8 illustrates, in side view, a portion of an intermediate body element 802. The intermediate body element 802 includes multiple conductors, such as conductor 840, arranged into a single-layer coil. The conductors 840 may be coiled over a cylindrical element 880 (e.g., a liner disposed over a removable mandrel, or the like). In at least some embodiments, the coiled conductors 840 are encased in an outer jacket 810, as discussed above with reference to outer jacket 410 of FIGS. 4A-4D.

The single-layer coil of the conductors 840 may extend along the entire length of the intermediate body element 802, or solely along one or more portions thereof. The intermediate body element 802 defines the conductors 840 twisted such that the individual conductors 840 form a helix around the cylindrical element 880. The conductors 840 can extend in either clockwise or counter-clockwise directions.

Turning to FIG. 9, in at least some embodiments the conductors are formed into multi-layer coils. FIG. 9 illustrates, in side view, a portion of an intermediate body element 902. The intermediate body element 902 includes two sets of multiple conductors 940a and 940b configured into a multi-layer coil. As described above with reference to FIG. 8, the conductors may be coiled over a cylindrical element 980 and may, optionally, be encased in an outer jacket 910. The multi-layer coil of conductors 940a and 940b may extend along the entire length of intermediate body element 902, or solely along one or more portions thereof.

The intermediate body element 902 defines an inner layer of conductors 940a. In some embodiments, the conductors 940a include 4 to 8 conductors, twisted such that the individual conductors 940a form a helix in a clockwise (or counterclockwise) direction around the cylindrical element 980. The intermediate body element 902 also defines an outer layer of conductors 940b disposed over the inner layer of conductors 940a. In some embodiments, the conductors 940b include 4 to 8 conductors, twisted in an opposite direction from the conductors 940a such that the individual conductors 940b form a helix in a counterclockwise (or clockwise) direction around the inner layer of the conductors 940a.

It may be desirable to design the lead extension 400 to reduce, or even prevent, undesired heating and other ill effects caused by common-mode coupling of transient RF pulses. Conventional implanted electrical stimulation systems are often incompatible with the magnetic resonance imaging (“MRI”) due to the large radio frequency (“RF”) pulses used during MRI. The RF pulses can generate transient signals in the conductors, electrodes, connector contacts, and terminals of an implanted lead and lead extension. These signals can have deleterious effects including, for example, unwanted heating of the tissue causing tissue damage, induced currents in the lead and lead extension, and premature failure of electronic components.

The helical shape of the coaxial conductors 940a and 940b may reduce heating of the lead extension during exposure to MRI. In addition, the pitch and direction for the inner conductors 940a can be different from the pitch of the outer conductors 940b and can be adjusted to reduce, or even cancel, the common-mode current. In addition, the helical configurations of lead extensions 802 and 902 provide flexibility and stretchability to the lead extension 400, thereby increasing the strain relief.

Referring to both FIGS. 8 and 9, the conductors 840, 940a, and 940b can have any suitable pitch. Further, the pitch can be either constant or variable. In some embodiments, the pitch is at least 0.04 turns per cm. In some embodiments, the pitch is no less than 0.1 turns per cm. In some embodiments, the pitch is at least 0.2 turns per cm. In some embodiments, the pitch is at least 0.25 turns per cm. In some embodiments, the pitch is at least 0.8 turns per cm. In some embodiments, the pitch is at least 0.4 turns per cm and no greater than 0.8 turns per cm. In some embodiments, the pitch is at least 0.1 turns per cm and no greater than 0.6 turns per cm. In some embodiments, the pitch is at least 0.1 turns per cm and no greater than 0.4 turns per cm. In some embodiments, the pitch is at least 0.2 turns per cm and no greater than 0.4 turns per cm. In some embodiments, the pitch is 0.3 turns per cm.

Turning to FIGS. 10A-10D, the conductors extending along the single intermediate body element are coupled on one end to connector contacts that are each disposed along one of multiple different connector housings, and on an opposing end to terminals that are each disposed along one of multiple different proximal tails. Thus, in the case of the terminals, some of the conductors extending along the intermediate body element are routed to one of the proximal tails, while some other of the conductors extending along the intermediate body element are routed to another one of the proximal tails. The conductors are similarly routed between the connector contacts disposed in different connector housings.

It may be advantageous to facilitate routing of the conductors by disposing multi-lumen tubing along one or more ends of the intermediate body element. FIG. 10C illustrates, in side view, the intermediate body element 802 with multi-lumen tubing 412 disposed along opposing ends of the intermediate body element 802. Each conductor of the conductors 840 extending along the intermediate body element 802 is routed to one or more connector contacts disposed along one of the multiple connector housings. Additionally, each conductor of the conductors 840 extending along the intermediate body element 802 is routed to one or more terminals disposed along one of the multiple proximal tails. The conductors can be routed in any suitable manner between the intermediate body element and the connector assembly 420, between the intermediate body element and the proximal tails 414a and 414b, or both.

FIGS. 10B-10D illustrate, in transverse cross-section, several different embodiments of the multi-lumen tubing 412. The multi-lumen tubing 412 includes one or more dividers 1092 that form multiple discrete conductor lumens 1094. Any suitable number of conductor lumens 1094 may be formed by the dividers 1092 including, for example, two, three, four, five, six, seven, eight, or more conductor lumens 1094. In at least some embodiments, each of the conductor lumens 1094 is configured to receive multiple conductors.

In FIG. 10B, the multi-lumen tubing 412 is divided into four sector-shaped conductor lumens 1094. In FIG. 10C, the multi-lumen tubing 412 is divided into three sector-shaped conductor lumens 1094. Additionally, FIG. 10C shows an optional stylet lumen 1096 formed by the dividers 1092. In FIG. 10D, the multi-lumen tubing 412 is divided into multiple conductor lumens 1094 formed as concentric circles. In at least some embodiments, the concentric circles are connected to one another via additional dividers 1092.

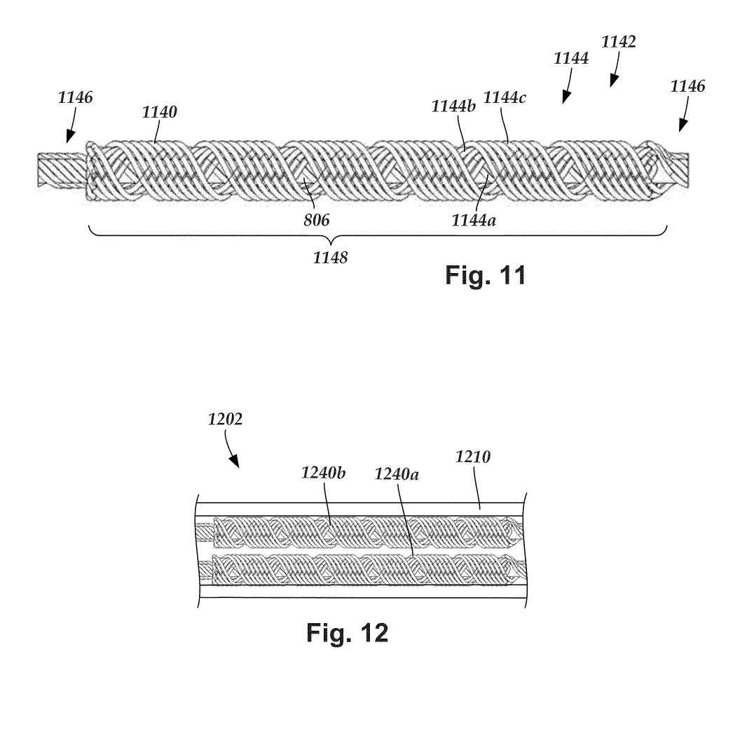

Turning to FIG. 11, as discussed above with reference to FIG. 9 certain arrangements of conductors can reduce one or more of the ill effects associated with common-mode coupling of current. FIG. 11 illustrates, in side view, conductors 1140 arranged into common-mode current suppression units to reduce the effects of the common-mode current generated in the conductors 1140 during, for example, an MRI procedure. The conductors 1140 have a winding geometry 1142 that includes a number of common-mode current suppression units (“units”) 1144 arranged in series. Examples of electrical stimulation systems with leads having conductors formed into units are found in, for example, U.S. Patent Application Publication Nos. 2010/0076508; 2010/0094364; and 2010/0256693, and U.S. patent application Ser. Nos. 12/494,086; 12/499,626; and 12/544,903, all of which are incorporated by reference.

Each unit 1144 includes at least three conductor segments that at least partially overlap one another to form a multi-layer region. First, each unit 1144 includes a first conductor segment 1144a that extends in a first direction along a longitudinal length of an elongated member (e.g., a lead extension) from a beginning point to a first position. Second, each unit 1144 includes a second conductor segment 1144b that extends from the first position back towards (and possibly past) the beginning point to a second position. Third, each unit 1144 includes a third conductor segment 1144c that extends in the first direction from the second position to an endpoint. In at least some embodiments, the first position is between the second position and the endpoint. In at least some embodiments, the second position is between the beginning point and the first position. In at least some embodiments, the unit 1144 includes a single-layer region flanking at least one end of the multi-layer region.

The units 1144 can be electrically continuous such that the endpoint of a first unit 1144 is the beginning point of the next consecutive unit 1144. At least one of the beginning points for the series of units 1144 can be a terminal or an electrode (or other conductive contact). Likewise, at least one of the endpoints for the series of units 1144 can be a terminal or an electrode (or other conductive contact). In preferred embodiments, each of the conductor segments (i.e. 1144a, 1144b, and 1144c) is coiled.

In some embodiments, at least one of the first, second, or third conductor segments (1144a, 1144b, and 1144c) is substantially straight. In at least some embodiments, the first and third conductor segments (1144a and 1144c) are substantially straight and the second conductor segment 1144b is coiled. In some other embodiments, all three conductor segments (1144a, 1144b, and 1144c) are substantially straight. It will be understood that the term “substantially straight conductor segment” means that the conductor segment is not coiled. A “substantially straight conductor segment” may be curved (but does not make a full revolution around a circumference of the cylindrical element 806 along a length of the conductor segment), particularly when the lead extension 400 itself is curved.

In some embodiments, the conductor segments are all formed from the same length of conductive material (e.g., wire or the like). The conductors may have a single filament or be multi-filar. In preferred embodiments, the conductors are multi-filar. In some embodiments, two or more of the conductor segments can be individual pieces of conductive material that are electrically coupled (e.g., soldered or welded) together.

In some embodiments, the length of conductor used in the second conductor segment is at least 1.5, 1.75, 1.9, 2, 2.1, 2.25, or 2.5 times the length of either the first conductor segment or the third conductor segment. It will be recognized, however, that this ratio of conductor-segment lengths may vary among embodiments, particularly if the thickness of the conductor or thickness of conductor insulation disposed around the conductors is different for the different segments.

Many different numbers of units 1144 may be disposed along longitudinal lengths of the conductors 1140 including, for example, two, three, four, five, six, seven, eight, nine, ten, twelve, fifteen, twenty, twenty-five, thirty, forty, fifty, or more units 1144. It will be understood that many other numbers of units 1144 may be employed as well. When a number of units 1144 are coupled in series along a longitudinal length of one or more conductors, the number of units 1144 form a repeating series of single-layer regions, such as the single-layer regions 1146, separated from one another by a multi-layer region, such as the multi-layer region 1148.

Turning to FIG. 12, in at least some embodiments the conductors extending along the intermediate body element are arranged into units. In at least some embodiments, multiple groupings, or sets, of conductors are extended along the intermediate body element, where each of the sets of conductors is arranged into multiple axially-spaced-apart units, and where each of the sets of conductors is disposed along the intermediate body element in a side-by-side arrangement with the remaining sets of conductors.

FIG. 12 illustrates, in longitudinal cross-section, one embodiment of a portion of an intermediate body element 1202 having an optional outer jacket 1210. FIG. 12 also illustrates, in side view, one embodiment of two sets of conductors 1240a and 1240b extending along the intermediate body element 1202 such that each of the sets of conductors 1240a and 1240b is arranged into axially-spaced-apart units, as described above with reference to FIG. 11. In FIG. 12, the optional outer jacket 1210 is shown collectively disposed around both sets of conductors 1240a and 1240b. In at least some embodiments, the conductors 1240a couple the connector contacts (434a of FIG. 4B) to the terminals (416a of FIG. 4A), and the conductors 1240b couple the connector contacts (434b of FIG. 4B) to the terminals (416b of FIG. 4A)

In one narrow embodiment, an electrical stimulation system, including the disclosed lead extension 400, can be implanted into a patient (e.g., in the patient's brain) by advancing a first stimulation lead to a target stimulation location within the patient. The first stimulation lead includes multiple electrodes disposed along the distal end portion of the lead. A proximal end portion of the lead includes multiple terminals electrically connected to the electrodes. A second stimulation lead is advanced to a target stimulation location within the patient. The second stimulation lead, likewise, includes multiple electrodes disposed along the distal end portion of the lead. A proximal end portion of the lead includes multiple terminals electrically connected to the electrodes. A medical practitioner electrically couples the terminals of the first and the second leads to the connector assembly of the lead extension. The medical practitioner electrically couples the corresponding terminals, disposed along the proximal tails 414a and 414b of the lead extension, to a control unit. The physician may, optionally, anchor the lead extension to patient tissue using, for example, sutures.

It will be understood that the embodiments of the lead extension described above can be used in any stimulation procedure for muscular or nervous tissue, such as deep brain stimulation, spinal cord stimulation, or the like. Further, the lead extension described above can be used in any industrial or medical application where strain relief and MRI-safety may be beneficial.

The above specification, examples, and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An implantable lead extension comprising:
an intermediate body element having a proximal end portion, a distal end portion, and a longitudinal length;
a plurality of proximal tails attached to the proximal end portion of the intermediate body element;
a different plurality of terminals disposed along each of the plurality of proximal tails;
a connector assembly attached to the distal end portion of the intermediate body element, the connector assembly comprising a plurality of connectors each configured and arranged for electrically coupling with a different stimulation lead, each of the plurality of connectors comprising
a connector housing defining a port for receiving a proximal end portion of a stimulation lead, and
a plurality of connector contacts disposed in the connector housing, the connector contacts configured and arranged to couple to terminals of the stimulation lead when the proximal end portion of the stimulation lead is received by the port; and
a plurality of conductors extending along the longitudinal length of the intermediate body element and electrically coupling the plurality of connector contacts to the plurality of terminals.

2. The implantable lead extension of claim 1, wherein the connector housings of the plurality of connectors are attached directly to one another along the longitudinal length of the connector assembly.

3. The implantable lead extension of claim 1, further comprising a conductor-carrying element disposed along the intermediate body element, the conductor-carrying element defining a plurality of conductor lumens, wherein the plurality of conductors extend along the longitudinal length of the intermediate body element within the plurality of conductor lumens.

4. The implantable lead extension of claim 1, wherein the plurality of conductors are formed into a single-layer coil as the plurality of conductors extend along the longitudinal length of the intermediate body element.

5. The implantable lead extension of claim 1, wherein the plurality of conductors are formed into a multi-layer coil as the plurality of conductors extend along the longitudinal length of the intermediate body element.

6. The implantable lead extension of claim 1, wherein the plurality of conductors are formed into a plurality of axially-spaced-apart common-mode current-suppression units as the plurality of conductors extend along the longitudinal length of the intermediate body element, each of the common-mode current-suppression units comprising:
a first conductor segment extending along the intermediate body element from a beginning point to a first position;
a second conductor segment extending along the intermediate body element from the first position to a second position; and
a third conductor segment extending along the intermediate body element from the second position to an endpoint;
wherein the first position is between the second position and the endpoint, and the second position is between the beginning point and the first position.

7. The implantable lead extension of claim 6, wherein the plurality of conductors are formed into a first grouping of conductors arranged into a first plurality of common-mode current-suppression units, and a second grouping of conductors arranged into a second plurality of common-mode current-suppression units.

8. The implantable lead extension of claim 7, wherein the first grouping of conductors and the second grouping of conductors extend parallel to each other along the longitudinal length of the intermediate body element.

9. The implantable lead extension of claim 1, further comprising a bellows element disposed between the connector assembly and the distal end portion of the intermediate body element, the bellows element providing strain relief for the connector assembly.

10. The implantable lead extension of claim 1, further comprising an outer jacket disposed over the plurality of conductors extending along the intermediate body element.

11. The implantable lead extension of claim 1, wherein the outer jacket comprises polyethylene terephthalate.

12. A lead extension assembly comprising:
the implantable lead extension of claim 1; and
at least one anchoring unit configured and arranged to facilitate anchoring of the implantable lead extension to patient tissue.

13. The implantable lead extension of claim 12, wherein the at least one anchoring unit comprises a suture sleeve.

14. The implantable lead extension of claim 12, wherein the at least one anchoring unit comprises an eyelet attached to at least one of the connector assembly, the intermediate body element, or the plurality of proximal tails.

15. A lead assembly comprising:
a first stimulation lead comprising
a first lead body having a distal end portion and an opposing proximal end portion;
a plurality of first terminals disposed along the proximal end portion of the lead body,
a plurality of first electrodes disposed along the distal end portion of the lead body, and
a plurality of conductors electrically coupling the plurality of first terminals to the plurality of first electrodes; and
the lead extension of claim 1;
wherein the first stimulation lead is configured and arranged for coupling to the connector assembly of the lead extension.

16. The lead assembly of claim 15, further comprising:
a second stimulation lead comprising
a second lead body having a distal end portion and an opposing proximal end portion;
a plurality of second terminals disposed along the proximal end portion of the lead body,
a plurality of second electrodes disposed along the distal end portion of the lead body, and
a plurality of conductors electrically coupling the plurality of second terminals to the plurality of second electrodes;
wherein second stimulation lead is configured and arranged for coupling to the connector assembly of the lead extension concurrently with the first stimulation lead.

17. A deep brain stimulation system comprising:
the lead assembly of claim 15; and
an implantable pulse generator configured and arranged to electrically couple to the first stimulation lead of the lead assembly.

18. A method for implanting an electrical stimulation system, the method comprising:
providing the lead extension of claim 1;
advancing a first stimulation lead into a skull of a patient, the first stimulation lead comprising a plurality of first electrodes disposed along a distal end portion of the first stimulation lead and a first plurality of terminals disposed along a proximal end portion of the first stimulation lead;
advancing a second stimulation lead into a skull of a patient, the second stimulation lead comprising a plurality of second electrodes disposed along a distal end portion of the second stimulation lead and a second plurality of terminals disposed along a proximal end portion of the second stimulation lead;
electrically coupling the first plurality of terminals of the first stimulation lead to the connector assembly of the lead extension;
electrically coupling the second plurality of terminals of the second stimulation lead to the connector assembly of the lead extension; and
coupling the plurality of terminals of each of the plurality of proximal tails of the lead extension to an implantable pulse generator.

19. The method of claim 18, further comprising anchoring the lead extension to patient tissue.

20. The method of claim 19, wherein anchoring the lead extension to patient tissue comprises anchoring the lead extension to patient tissue using at least one suture sleeve.
